(19) United States
(12) Patent Application Publication
(10) Pub. No.: US 2023/0236181 A1
(43) Pub. Date: Jul. 27, 2023

(54) LIQUID REFINING APPARATUS AND DIAGNOSIS SYSTEM INCLUDING THE SAME

(71) Applicant: SPECLIPSE, INC., Gyeonggi-do (KR)

(72) Inventors: Sung Hyun PYUN, Seoul (KR); Wan Ki MIN, Seoul (KR)

(21) Appl. No.: 17/702,898

(22) Filed: Mar. 24, 2022

(30) Foreign Application Priority Data
Jan. 27, 2022 (KR) 10-2022-0012314
Jan. 27, 2022 (KR) 10-2022-0012316

(51) Int. Cl. G01N 33/543; B01L 3/00; G01N 21/65; B01D 21/26; B01D 21/28; B01D 21/00

(52) U.S. Cl. CPC G01N 33/54373; B01D 21/0018; B01D 21/265; B01D 21/283; B01L 3/502753; G01N 21/658; B01L 2200/0647; B01L 2300/087; B01L 2300/0654; B01L 2300/0681; B01L 2300/0883; G01N 2201/06113

(57) ABSTRACT

A liquid refining apparatus is disclosed. The liquid refining apparatus includes a substrate, a loader which is formed on the substrate and configured to receive a first liquid, a filter which is configured to reduce a concentration of at least one substance contained in the first liquid to obtain a second liquid with a reduced concentration of the at least one substance, a reactor which is configured to mix the second liquid with a reactant for target substance detection to obtain a third liquid containing, among a plurality of substances contained in the second liquid, a first substance which undergoes a predetermined reaction with the reactant and a second substance which does not undergo the predetermined reaction with the reactant, and a separator which is configured to separate the first substance and the second substance.

### CROSS-REFERENCE TO RELATED APPLICATION

This application claims priority to and the benefit of Korean Patent Application No. 2022-0012314, filed on Jan. 27, 2022, and Korean Patent Application No. 2022-0012316, filed on Jan. 27, 2022, the disclosure of which is incorporated herein by reference in its entirety.

### FIELD

Embodiments relate to a liquid refining apparatus.

Embodiments relate to a medical diagnosis apparatus.

Embodiments relate to a medical diagnosis system.

### BACKGROUND

Research on technology for diagnosing a subject’s health status or the presence or absence of disease based on a liquid (e.g., blood or urine) collected from the subject is being actively carried out. For example, the technology may analyze spectral information on the liquid to obtain a diagnosis result for the subject. Generally, the technology obtains a diagnosis result based on spectral information on blood in which an antigen-antibody reaction has occurred.

However, various components other than antigens, such as blood cell components, are present in blood, causing the spectral information to contain an error. Therefore, in order to obtain an accurate diagnosis result, there is a need for a technology capable of preventing an occurrence of an error in the spectral information or compensating for the error.

### SUMMARY

The present disclosure is directed to providing a liquid refining apparatus capable of improving the accuracy of a diagnosis result for a subject.

The present disclosure is also directed to providing a diagnosis apparatus capable of compensating for an error in spectral information on a target substance.

The objectives of the present disclosure are not limited to those mentioned above, and other unmentioned objectives should be clearly understood by those of ordinary skill in the art to which the present disclosure pertains from the description below.

One exemplary embodiment of the present disclosure provides a liquid refining apparatus including: a substrate; a loader which is formed on the substrate and configured to receive a first liquid; a filter which is configured to reduce a concentration of at least one substance contained in the first liquid to obtain a second liquid with a reduced concentration of the at least one substance; a reactor which is configured to mix the second liquid with a reactant for target substance detection to obtain a third liquid containing, among a plurality of substances contained in the second liquid, a first substance which undergoes a predetermined reaction with the reactant and a second substance which does not undergo the predetermined reaction with the reactant; and a separator which is configured to separate the first substance and the second substance.

One exemplary embodiment of the present disclosure provides a diagnosis system including a liquid refining apparatus, a liquid information obtaining apparatus, and a diagnosis apparatus, wherein the liquid refining apparatus includes a substrate, a loader which is formed on the substrate and configured to receive a first liquid collected from a subject, a filter which is configured to reduce a concentration of at least one substance contained in the first liquid to obtain a second liquid with a reduced concentration of the at least one substance, a reactor which is configured to mix the second liquid with a reactant for target substance detection to obtain a third liquid containing, among a plurality of substances contained in the second liquid, a first substance which undergoes a predetermined reaction with the reactant and a second substance which does not undergo the predetermined reaction with the reactant, and a separator which is configured to separate the first substance and the second substance, the liquid information obtaining apparatus irradiates the first substance with light to obtain a Raman signal for the first substance, and the diagnosis apparatus obtains a diagnosis result for the subject based on the Raman signal.

One exemplary embodiment of the present disclosure provides a diagnosis apparatus including: a memory configured to store at least one instruction; and a processor, wherein, by executing the at least one instruction, the processor obtains information on a first spectrum that corresponds to a first liquid which is collected from a subject and contains a first target substance and information on a second spectrum that corresponds to a second liquid which contains the first target substance which is, as a first reactant for detection of the first target substance is added to the first liquid, bound to the first reactant, obtains first concentration information of the first target substance based on the information on the first spectrum and the information on the second spectrum, and obtains diagnosis information on the subject based on the first concentration information.

One exemplary embodiment of the present disclosure provides a control method of a diagnosis apparatus, the control method including: obtaining information on a first spectrum that corresponds to a first liquid which is collected from a subject and contains a first target substance and information on a second spectrum that corresponds to a second liquid which contains the first target substance which is, as a first reactant for detection of the first target substance is added to the first liquid, bound to the first reactant; obtaining first concentration information of the first target substance based on the information on the first spectrum and the information on the second spectrum; and obtaining diagnosis information on the subject based on the first concentration information.

The means for achieving the objectives of the present disclosure are not limited to those described above, and other unmentioned means should be clearly understood by those of ordinary skill in the art to which the present disclosure pertains from this specification and the accompanying drawings.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 is a block diagram illustrating a configuration of a diagnosis system according to an embodiment of the present disclosure;

FIG. 2 is a block diagram illustrating a configuration of a liquid refining apparatus according to an embodiment of the present disclosure;

FIG. 3 is a view of the liquid refining apparatus from the top according to an embodiment of the present disclosure;

FIG. 4 is a view of the liquid refining apparatus from the bottom according to an embodiment of the present disclosure;

FIG. 5 is a view of a liquid refining apparatus from the top according to an embodiment of the present disclosure;

FIG. 6 is a view illustrating a filter according to an embodiment of the present disclosure;

FIG. 7 is a view illustrating a separator according to an embodiment of the present disclosure;

FIG. 8 is a view for describing a liquid information obtaining method of a liquid information obtaining apparatus according to an embodiment of the present disclosure;

FIG. 9 is a block diagram illustrating a configuration of a diagnosis apparatus according to an embodiment of the present disclosure;

FIG. 10 is a view illustrating information on a spectrum according to an embodiment of the present disclosure;

FIG. 11 is a view for describing a method of obtaining concentration information according to a first embodiment of the present disclosure;

FIG. 12 is a view for describing a learning method of a first neural network model according to an embodiment of the present disclosure;

FIG. 13 is a view for describing a method of obtaining concentration information according to a second embodiment of the present disclosure;

FIG. 14 is a view for describing a method of obtaining concentration information according to a third embodiment of the present disclosure;

FIG. 15 is a view for describing a method of obtaining concentration information according to a fourth embodiment of the present disclosure;

FIG. 16 is a view for describing a method of obtaining concentration information according to an embodiment of the present disclosure;

FIG. 17 is a view for describing a method of obtaining concentration information according to an embodiment of the present disclosure;

FIG. 18 is a view for describing a method of obtaining concentration information according to an embodiment of the present disclosure; and

FIG. 19 is a flowchart illustrating a control method of the diagnosis apparatus according to an embodiment of the present disclosure.

### DETAILED DESCRIPTION

Terms used herein will be briefly described, and the present disclosure will be described in detail.

The terms used in the embodiments of the present disclosure are general widely-used terms selected in consideration of functions in the present disclosure, but the terms may vary depending on the intention or practice of one of ordinary skill in the art or the advent of new technology. Also, there are some terms arbitrarily selected by the applicant, and in this case, the meaning of the terms will be specifically described in the corresponding part of the description of the disclosure. Therefore, the terms used herein should be defined based on the meanings thereof and the entire content herein instead of being defined simply based on the names of the terms.

Since various modifications may be made to the embodiments of the present disclosure and the present disclosure may have various embodiments, specific embodiments will be illustrated in the drawings and described in detail in the following detailed description. However, this is not intended to limit the scope of the present disclosure to the specific embodiments, and all modifications, equivalents, and substitutes included in the disclosed spirit and technical scope should be understood as belonging to the scope of the present disclosure. In describing the embodiments, when detailed description of a related known art is determined as having the possibility of obscuring the gist of the present disclosure, the detailed description thereof will be omitted.

Terms such as first and second may be used to describe various elements, but the elements are not limited by the terms. The terms are only used for the purpose of distinguishing one element from another element.

A singular expression includes a plural expression unless the context clearly indicates otherwise. In this application, terms such as “include” or “have” should be understood as specifying that features, number, steps, operations, elements, components, or combinations thereof are present and not as precluding the possibility of the presence or addition of one or more other features, numbers, steps, operations, elements, components, or combinations thereof in advance.

Hereinafter, embodiments of the present disclosure will be described in detail with reference to the accompanying drawings to allow those of ordinary skill in the art to which the present disclosure pertains to easily carry out the present disclosure. However, the present disclosure may be implemented in various different forms and is not limited to the embodiments described herein. Also, parts unrelated to the description have been omitted from the drawings for clarity of the description of the present disclosure, and like parts are denoted by like reference numerals throughout.

One exemplary embodiment of the present disclosure provides a liquid refining apparatus including: a substrate; a loader which is formed on the substrate and configured to receive a first liquid; a filter which is configured to reduce a concentration of at least one substance contained in the first liquid to obtain a second liquid with a reduced concentration of the at least one substance; a reactor which is configured to mix the second liquid with a reactant for target substance detection to obtain a third liquid containing, among a plurality of substances contained in the second liquid, a first substance which undergoes a predetermined reaction with the reactant and a second substance which does not undergo the predetermined reaction with the reactant; and a separator which is configured to separate the first substance and the second substance.

The first liquid may contain blood in an undiluted state, and the at least one substance may include blood cell components.

The predetermined reaction may include an antigen-antibody reaction.

The liquid refining apparatus may further include an anticoagulator configured to add an anticoagulant to the first liquid.

The reactor may include a reactant storage configured to store the reactant and a zigzag mixing channel configured to increase a mixing rate of the second liquid and the reactant.

The reactant may be bound to metal nanoparticles which are bound to Raman active particles.

The liquid refining apparatus may further include: a first chamber formed on the substrate and connected to the loader to store the first liquid; a second chamber formed on the substrate and connected to the filter to store the second liquid; and a third chamber formed on the substrate and connected to the separator to store the first substance.

The liquid refining apparatus may further include a concentrator configured to perform a concentration process on the third liquid, and the concentration process may include at least one of drying, heating, and baking.

The liquid refining apparatus may further include a pumper configured to move the first liquid, the second liquid, and the third liquid, and the pumper may include at least one of a pneumatic pump, a vibration pump, a mechanical pump, and a capillary pump.

The reactor may include a first reactor which includes a first reactant storage configured to store a first reactant for first target detection and a first mixing channel configured to increase a mixing rate of the second liquid and the first reactant, a second reactor which includes a second reactant storage configured to store a second reactant for second target detection and a second mixing channel configured to increase a mixing rate of the second liquid and the second reactant, a first channel which is configured to transfer the second liquid to the first reactor, and a second channel which is configured to transfer the second liquid to the second reactor.

The first channel and the second channel may have a structure branched from an output end of the filter.

The separator may include a first separator which is connected to an output end of the first reactor and configured to separate, among a plurality of substances contained in the third liquid, a third substance which undergoes an antigen-antibody reaction with the first reactant from another substance, and a second separator which is connected to an output end of the second reactor and configured to separate, among the plurality of substances contained in the third liquid, a fourth substance which undergoes an antigen-antibody reaction with the second reactant from another substance.

The filter may include a lateral cavity acoustic transducer (LCAT) formed to protrude outward from a filter channel through which the first liquid flows.

The separator may separate the first substance and the second substance according to the molecular weight based on a sound wave and may include a first outlet channel configured to move the first substance and a second outlet channel configured to move the second substance.

One exemplary embodiment of the present disclosure provides a diagnosis system including a liquid refining apparatus, a liquid information obtaining apparatus, and a diagnosis apparatus, wherein the liquid refining apparatus includes a substrate, a loader which is formed on the substrate and configured to receive a first liquid collected from a subject, a filter which is configured to reduce a concentration of at least one substance contained in the first liquid to obtain a second liquid with a reduced concentration of the at least one substance, a reactor which is configured to mix the second liquid with a reactant for target substance detection to obtain a third liquid containing, among a plurality of substances contained in the second liquid, a first substance which undergoes a predetermined reaction with the reactant and a second substance which does not undergo the predetermined reaction with the reactant, and a separator which is configured to separate the first substance and the second substance, the liquid information obtaining apparatus irradiates the first substance with light to obtain a Raman signal for the first substance, and the diagnosis apparatus obtains a diagnosis result for the subject based on the Raman signal.

One exemplary embodiment of the present disclosure provides a diagnosis apparatus including: a memory configured to store at least one instruction; and a processor, wherein, by executing the at least one instruction, the processor obtains information on a first spectrum that corresponds to a first liquid which is collected from a subject and contains a first target substance and information on a second spectrum that corresponds to a second liquid which contains the first target substance which is, as a first reactant for detection of the first target substance is added to the first liquid, bound to the first reactant, obtains first concentration information of the first target substance based on the information on the first spectrum and the information on the second spectrum, and obtains diagnosis information on the subject based on the first concentration information.

The first liquid may not contain the first reactant.

The information on the second spectrum may include a peak value of the second spectrum, and by executing the at least one instruction, the processor may, based on a table in which peak values of spectra and pieces of concentration information are matched and which is pre-stored in the memory, obtain concentration information that corresponds to the peak value of the second spectrum, may input the information on the first spectrum into a first neural network model to obtain a coefficient for compensating for the concentration information, and may compensate for the concentration information based on the coefficient to obtain the first concentration information.

By executing the at least one instruction, the processor may input the information on the first spectrum and the information on the second spectrum into a second neural network model to obtain the first concentration information.

By executing the at least one instruction, the processor may input the information on the second spectrum into a second neural network model to obtain a feature vector and may input the information on the first spectrum and the feature vector into a third neural network model to obtain the first concentration information.

By executing the at least one instruction, the processor may input the information on the first spectrum into a second neural network model to obtain a first feature vector, input the information on the second spectrum into the second neural network model to obtain a second feature vector, and input the first feature vector and the second feature vector into a fourth neural network model to obtain the first concentration information.

By executing the at least one instruction, the processor may input the information on the first spectrum and the first concentration information into a fifth neural network model to obtain the diagnosis information.

By executing the at least one instruction, the processor may obtain information on a third spectrum that corresponds to a third liquid which contains a second target substance which is, as a second reactant for detection of the second target substance contained in the first liquid is added, bound to the second reactant, may obtain second concentration information of the second target substance based on the information on the first spectrum and the information on the third spectrum, and may input the information on the first spectrum, the first concentration information, and the second concentration information into the fifth neural network model to obtain the diagnosis information.

By executing the at least one instruction, the processor may input the information on the first spectrum and the information on the second spectrum into a sixth neural network model to obtain the diagnosis information.

One exemplary embodiment of the present disclosure provides a control method of a diagnosis apparatus, the control method including: obtaining information on a first spectrum that corresponds to a first liquid which is collected from a subject and contains a first target substance and information on a second spectrum that corresponds to a second liquid which contains the first target substance which is, as a first reactant for detection of the first target substance is added to the first liquid, bound to the first reactant; obtaining first concentration information of the first target substance based on the information on the first spectrum and the information on the second spectrum; and obtaining diagnosis information on the subject based on the first concentration information.

The obtaining of the first concentration information may include, based on a table in which peak values of spectra and pieces of concentration information are matched and which is pre-stored in the memory, obtaining concentration information that corresponds to a peak value of the second spectrum, inputting the information on the first spectrum into a first neural network model to obtain a coefficient for compensating for the concentration information, and compensating for the concentration information based on the coefficient to obtain the first concentration information.

The obtaining of the first concentration information may include inputting the information on the first spectrum and the information on the second spectrum into a second neural network model to obtain the first concentration information.

The obtaining of the first concentration information may include inputting the information on the second spectrum into a second neural network model to obtain a feature vector, and inputting the information on the first spectrum and the feature vector into a third neural network model to obtain the first concentration information.

The obtaining of the first concentration information may include inputting the information on the first spectrum into a second neural network model to obtain a first feature vector, inputting the information on the second spectrum into the second neural network model to obtain a second feature vector, and inputting the first feature vector and the second feature vector into a fourth neural network model to obtain the first concentration information.

The obtaining of the diagnosis information may include inputting the information on the first spectrum and the first concentration information into a fifth neural network model to obtain the diagnosis information.

The control method may further include: obtaining information on a third spectrum that corresponds to a third liquid which contains a second target substance which is, as a second reactant for detection of the second target substance contained in the first liquid is added, bound to the second reactant; and obtaining second concentration information of the second target substance based on the information on the first spectrum and the information on the third spectrum, and the obtaining of the diagnosis information may include inputting the information on the first spectrum, the first concentration information, and the second concentration information into the fifth neural network model to obtain the diagnosis information.

The obtaining of the diagnosis information may include inputting the information on the first spectrum and the information on the second spectrum into a sixth neural network model to obtain the diagnosis information.

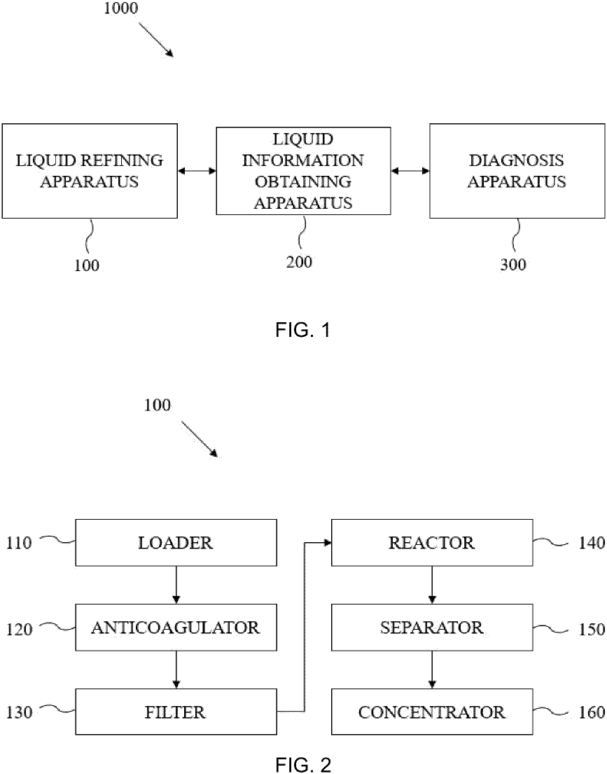

FIG. 1 is a block diagram illustrating a configuration of a diagnosis system according to an embodiment of the present disclosure.

Referring to FIG. 1, a diagnosis system 1000 may include a liquid refining apparatus 100, a liquid information obtaining apparatus 200, and a diagnosis apparatus 300.

The liquid refining apparatus 100 is an apparatus for separating a liquid. For example, the liquid refining apparatus 100 may separate blood cell components and plasma components of blood. Alternatively, the liquid refining apparatus 100 may separate a first blood to which a first reactant is added and a second blood to which a second reactant is added. The liquid may include at least one of urine, saliva, semen, sweat, tears, and cerebrospinal fluid of a subject. The subject may be a human or an animal.

The liquid refining apparatus 100 may be implemented using a biochip.

The liquid information obtaining apparatus 200 is an apparatus for obtaining liquid information, which is information on the liquid. The liquid information may include spectral information that corresponds to the liquid. The spectral information may indicate an intensity for each wavelength (or frequency). The spectral information (referred to as information on a spectrum) may include a Raman signal.

The liquid information obtaining apparatus 200 may obtain spectral information based on Raman scattering that occurs when light passes through a certain medium. For example, the liquid information obtaining apparatus 200 may irradiate a liquid obtained by the liquid refining apparatus 100 with a laser beam and obtain a laser beam scattered from fine particles in the liquid. The liquid refining apparatus 100 may, based on the scattered laser beam, obtain spectral information corresponding to the liquid. The spectral information, that is, wavelengths of a Raman signal and intensities thereof in wavelength bands, may vary according to components of the fine particles.

The liquid information obtaining apparatus 200 may include an intensity that corresponds to only a specific frequency of the spectral information. For example, in a case where Raman active molecules are bound to the fine particles, the liquid information obtaining apparatus 200 may obtain a peak value of the Raman signal at a frequency that corresponds to the Raman active molecules.

The liquid information obtaining apparatus 200 may include a light source configured to irradiate the liquid with light. The light source may irradiate a laser beam for inducing Raman scattering from the fine particles in the liquid.

The liquid information obtaining apparatus 200 may include an optical system. The optical system may include a configuration for delivering light irradiated from the light source to the liquid and sensing light scattered from the fine particles in the liquid. For example, the optical system may include at least one of a filter, a mirror, a lens, a slit, a lattice, and a sensor. The sensor may sense the light scattered from the fine particles in the liquid.

The liquid information obtaining apparatus 200 may be implemented using a spectrometer.

The diagnosis apparatus 300 is a configuration for obtaining various analysis data related to a liquid. The diagnosis apparatus 300 may be a medical diagnosis apparatus. For example, the diagnosis apparatus 300 may obtain identification information and concentration information on a plurality of substances constituting a liquid obtained by the liquid information obtaining apparatus 200. Also, the diagnosis apparatus 300 may obtain a concentration ratio of the plurality of substances contained in the liquid.

The diagnosis apparatus 300 may obtain various diagnosis results. For example, the diagnosis apparatus 300 may obtain a diagnosis result including a health status or the presence or absence of disease that corresponds to a subject. The diagnosis result may include identification information on a disease that the subject is expected to have and information on the probability that the subject has the corresponding disease.

The diagnosis apparatus 300 may be implemented using a server or a user terminal device.

FIG. 2 is a block diagram illustrating a configuration of the liquid refining apparatus according to an embodiment of the present disclosure.

Referring to FIG. 2, the liquid refining apparatus 100 may include a loader 110, an anticoagulator 120, a filter 130, a reactor 140, a concentrator 160, and a separator 150.

The loader 110 may include an inlet through which a liquid is injected and a channel configured to move the liquid. The loader 110 may receive a liquid through the inlet and deliver the received liquid to another configuration of the liquid refining apparatus 100. For example, the loader 110 may receive a first liquid and deliver a portion of the first liquid to the anticoagulator 120. Alternatively, the loader 110 may deliver a portion of the first liquid to a chamber.

The anticoagulator 120 is a configuration for performing an anticoagulation process on a liquid. The anticoagulator 120 may include a chamber in which an anticoagulant is stored. For example, the anticoagulator 120 may be disposed on a path through which the first liquid received by the loader 110 moves to the filter 130. The anticoagulator 120 may include an inlet through which the first liquid is input and an outlet through which the first liquid on which the anticoagulation process is performed is output. Alternatively, the anticoagulator 120 may include a pipe for injecting an anticoagulant into a channel through which the first liquid flows to the filter 130.

The filter 130 may receive the liquid on which the anticoagulation process is performed. The filter 130 may reduce a concentration of at least one component contained in the received liquid. For example, the at least one component may include blood cell components. The blood cell components may include red blood cells, white blood cells, and platelets.

The filter 130 may include a filter channel through which a liquid flows and a fluid structure for filtering at least one component contained in the liquid. The filter channel may receive the first liquid on which the anticoagulation process is performed by the anticoagulator 120. The fluid structure may include a protruding portion which is formed to protrude outward from the filter channel and includes an air bag. For example, the fluid structure may include a lateral cavity acoustic transducer (LCAT).

The filter 130 may filter the at least one component contained in the liquid based on vibration transmitted to the filter channel and the air bag. When vibration is transmitted to the filter channel and the air bag, a vortex of the liquid may be formed on an interface between the filter channel and the air bag. The vortex of the liquid may hold the at least one component contained in the liquid passing through the filter channel. Accordingly, the filter 130 may obtain a liquid with a reduced concentration of the at least one component. For example, the filter 130 may receive the first liquid on which the anticoagulation process is performed to filter blood cell components contained in the first liquid on which the anticoagulation process is performed. Accordingly, the filter 130 may obtain a second liquid with a reduced concentration of the blood cell components. The second liquid may be plasma or serum.

The reactor 140 may induce a predetermined reaction between a reactant for target substance detection and a target substance contained in a liquid. The target substance may include an antigen. The reactant may include an antibody. The reactant may be bound to metal (e.g., gold) nanoparticles to which Raman active molecules (or Raman reporters) are bound. The predetermined reaction is a chemical reaction and, for example, may include an antigen-antibody reaction. A plurality of metal nanoparticles may be bound to an antigen through an antibody. Accordingly, a single metal nanoparticle mass in which the plurality of metal nanoparticles are bound may be formed around the antigen.

The reactor 140 may cause the second liquid to react with a reactant to obtain a third liquid. The second liquid may contain a first substance which undergoes a predetermined reaction with the reactant and a second substance which does not undergo the predetermined reaction with the reactant. The third liquid may include the first substance that has undergone the predetermined reaction with the reactant and the second substance that has not undergone the predetermined reaction with the reactant.

The reactor 140 may include a reactant storage configured to store a reactant. The reactor 140 may include a mixing channel configured to increase a mixing rate of the second liquid and the reactant. For example, the mixing channel may have a zigzag shape.

The separator 150 is a configuration for separating a substance contained in a liquid. For example, the separator 150 may separate the first substance and the second substance contained in the third liquid. The separator 150 may separate the first substance and the second substance based on various methods. For example, the separator 150 may separate the first substance and the second substance according to the molecular weight based on a surface acoustic wave (SAW). The separator 150 may include a surface acoustic wave filter.

The separator 150 may include a channel for moving the separated plurality of substances. For example, the separator 150 may include a first outlet channel configured to move the first substance and a second outlet channel configured to move the second substance.

The concentrator 160 is a configuration for performing a concentration process on a liquid to increase a concentration of a target substance contained in the liquid. The concentration process may include at least one of drying, heating, and baking. Alternatively, the concentrator 160 may include a filter (e.g., a membrane filter) or a pipe configured to allow only a substance having a size smaller than a predetermined size to pass. Here, the concentrator 160 may allow passage of substances whose size is smaller than the size of first metal nanoparticles which are bound to a target substance through a reactant and may not allow passage of the first metal nanoparticles. Accordingly, the concentrator 160 may obtain a liquid with an increased concentration of the first metal nanoparticles. That is, the concentrator 160 may obtain a liquid with an increased concentration of the target substance.

The concentrator 160 may obtain a fourth liquid which contains the first substance separated by the separator 150. The fourth liquid may contain the first metal nanoparticles bound to the first substance through the antibody. The fourth liquid may contain a third substance (e.g., protein) whose molecular weight is higher than the molecular weight of the first metal nanoparticles or the second substance not precisely separated by the separator 150. Alternatively, the fourth liquid may contain second metal nanoparticles not bound to the first substance. The concentrator 160 may allow passage of at least a portion of the remaining substance excluding the first metal nanoparticles and not allow passage of the first metal nanoparticles to obtain a fifth liquid with an increased concentration of the first metal nanoparticles. Accordingly, the concentrator 160 may obtain the fifth liquid in which the concentration of the first substance is increased as compared to the fourth liquid.

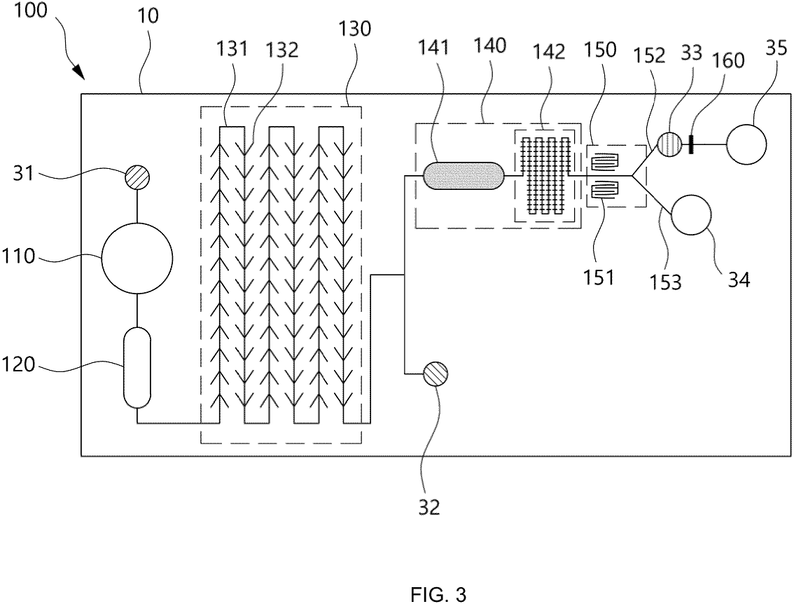

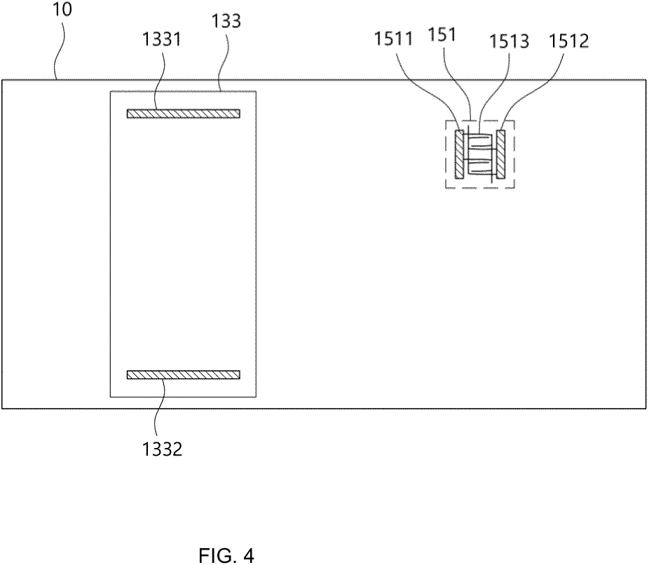

FIG. 3 is a view of the liquid refining apparatus from the top according to an embodiment of the present disclosure. FIG. 4 is a view of the liquid refining apparatus from the bottom according to an embodiment of the present disclosure.

Referring to FIG. 3, the liquid refining apparatus 100 may include a substrate 10, the loader 110, the anticoagulator 120, the filter 130, the reactor 140, the separator 150, the concentrator 160, and chambers 31, 32, 33, 34, and 35.

The loader 110 may be formed on the substrate 10. The loader 110 may receive the first liquid. The loader 110 may deliver a portion of the first liquid to a first chamber 31. The first chamber 31 may store the delivered first liquid.

The loader 110 may deliver the first liquid to the anticoagulator 120. The anticoagulator 120 may perform the anticoagulation process on the first liquid. The anticoagulator 120 may deliver the first liquid on which the anticoagulation process is performed to the filter 130.

The filter 130 may include a filter channel 131 through which the first liquid flows, a fluid structure 132 which is formed to protrude outward from the filter channel 131 and includes an air bag, and a first vibration generator 133 configured to provide a sound wave to the filter channel 131 and the fluid structure 132.

The filter 130 may filter at least one first component (e.g., blood cell component) contained in the first liquid based on the sound wave generated by the first vibration generator 133. The filter channel 131 and the fluid structure 132 may vibrate due to the sound wave generated by the first vibration generator 133. A vortex of the first liquid may be formed on an interface between the filter channel 131 and the air bag as the filter channel 131 and the fluid structure 132 vibrate. The at least one first component may be trapped in the formed vortex of the first liquid. Accordingly, the filter 130 may obtain the second liquid in which a concentration of the at least one first component is reduced as compared to the first liquid.

The filter 130 may move the first liquid and the second liquid based on the air bag included in the fluid structure 132. The air bag may repeat compression and expansion based on the wound wave generated by the first vibration generator 133. The air bag may pump the first liquid and the second liquid. The filter 130 may output the second liquid. The fluid structure 132 and the first vibration generator 133 may constitute a LCAT.

Referring to FIG. 4, the first vibration generator 133 may be disposed below the substrate 10. The first vibration generator 133 may include a plurality of electrodes 1331 and 1332. The plurality of electrodes 1331 and 1332 may be disposed to face each other. The plurality of electrodes 1331 and 1332 may be piezoelectric electrodes.

Referring back to FIG. 3, the filter 130 may deliver a portion of the second liquid to a second chamber 32. The second liquid may be blood obtained by removing blood cell components from the first liquid.

The filter 130 may deliver the second liquid to the reactor 140. The reactor 140 may include a reactant storage 141 configured to store a reactant bound to metal nanoparticles. Raman active molecules may be bound to the metal nanoparticles.

The second liquid may be mixed with the reactant while passing through the reactant storage 141. An antigen-antibody reaction may occur between a target substance (or a first substance) contained in the second liquid and the reactant. In this process, a metal nanoparticle mass in which one or more metal nanoparticles are agglomerated may be formed around the antigen.

The reactor 140 may include a zigzag mixing channel 142 configured to increase a mixing rate of the second liquid and the reactant. While the second liquid passes through the mixing channel 142, an antigen-antibody reaction may occur between the target substance and the reactant. Accordingly, the number of metal nanoparticle masses may be increased. Although not illustrated, the reactor 140 may include a LCAT configured to increase the mixing rate of the second liquid and the reactant.

The reactor 140 may obtain the third liquid based on the second liquid. The third liquid may contain a first substance in which a metal nanoparticle mass is formed through a reaction with the reactant and a plurality of second substances in which a metal nanoparticle mass is not formed due to not reacting with the reactant. The reactor 140 may deliver the third liquid to the separator 150.

The separator 150 may include a second vibration generator 151 configured to generate a sound wave, a first outlet channel 152, and a second outlet channel 153. The second vibration generator 151 may be provided below the substrate 10. The second vibration generator 151 may be an inter-digital transducer (IDT) electrode. The IDT electrode may include a plurality of bars 1511 and 1512 that face each other and a plurality of fingers 1513 that protrude from the plurality of bars 1511 and 1512. The sound wave may be SAW.

The separator 150 may separate the plurality of substances contained in the third liquid according to the molecular weight based on the sound wave. The separator 150 may generate vibration around a channel through which the liquid flows and may separate the plurality of substances based on the vibration. For example, the separator 150 may separate a substance bound to a target substance (e.g., metal nanoparticles bound to an antibody) and a substance not bound to the target substance. The separator 150 may filter the substance not bound to the target substance to obtain the fourth liquid. The fourth liquid may be a liquid obtaining by reducing a concentration of the substance not bound to the target substance in the third liquid.

The separator 150 may deliver the fourth liquid to a third chamber 33 through the first outlet channel 152. The separator 150 may deliver the substance not bound to the target substance to a fourth chamber 34 through the second outlet channel 153.

The concentrator 160 may perform a concentration process on the fourth liquid accommodated in the third chamber 33. The concentrator 160 may not allow passage of the metal nanoparticle mass containing the target substance among a plurality of substances contained in the fourth liquid and may only allow passage of the remaining substance. Accordingly, the concentrator 160 may obtain the fifth liquid in which a concentration of the metal nanoparticles or target substance is increased.

Meanwhile, in order to obtain a diagnosis result for a subject, a plurality of biomarkers (or target substances) may be necessary. Hereinafter, a liquid refining apparatus for refining a liquid containing a plurality of biomarkers will be described.

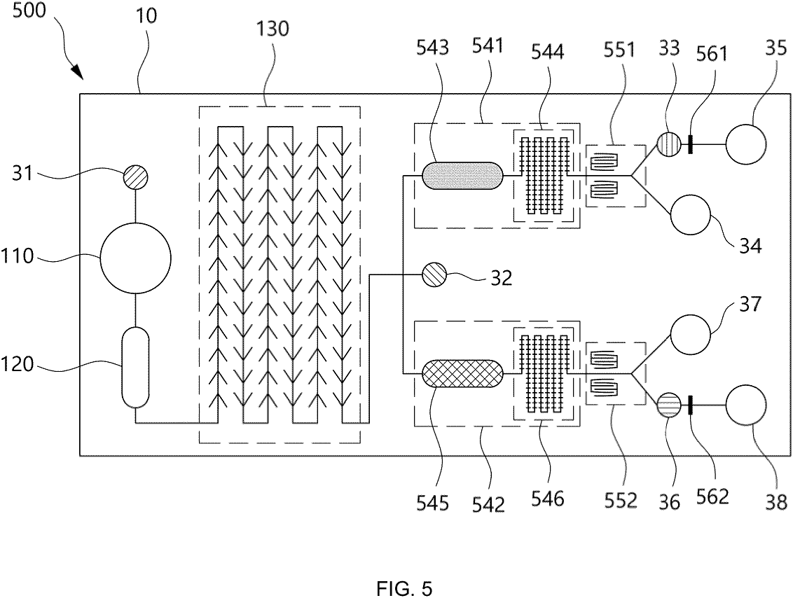

FIG. 5 is a view of a liquid refining apparatus from the top according to an embodiment of the present disclosure.

Referring to FIG. 5, a liquid refining apparatus 500 may include the loader 110, the anticoagulator 120, the filter 130, a first reactor 541, a second reactor 542, a first separator 551, a second separator 552, a first concentrator 561, and a second concentrator 562. Meanwhile, since the loader 110, the anticoagulator 120, and the filter 130 have been described above with reference to FIGS. 3 and 4, overlapping description thereof will be omitted. Also, a basic operation of the first reactor 541 and the second reactor 542 may be clearly understood through the reactor 140 of FIG. 3, a basic operation of the first separator 551 and the second separator 552 may be clearly understood through the separator 150 of FIG. 3, and a basic operation of the first concentrator 561 and the second concentrator 562 may be clearly understood through the concentrator 160 of FIG. 3. Therefore, description overlapping with the description of FIG. 3 will be omitted.

The first reactor 541 may include a first reactant storage 543 and a first reaction channel 544. The first reactant storage 543 may store a first reactant for detection of a first target substance. The first reaction channel 544 may increase a mixing rate of the first target substance and the first reactant. The first reaction channel 544 may induce an antigen-antibody reaction between the first target substance and the first reactant.

The second reactor 542 may include a second reactant storage 545 and a second reaction channel 546. The second reactant storage 545 may store a second reactant for detection of a second target substance. The second reaction channel 546 may increase a mixing rate of the second target substance and the second reactant. The second reaction channel 546 may induce an antigen-antibody reaction between the second target substance and the second reactant.

A first liquid passing through the first reactor 541 may contain a first metal nanoparticle mass formed due to a reaction between the first target substance and the first reactant and a plurality of first substances not bound to the first target substance. A second liquid passing through the second reactor 542 may contain a second metal nanoparticle mass formed due to a reaction between the second target substance and the second reactant and a plurality of second substances not bound to the second target substance.

The first separator 551 may separate the first metal nanoparticle mass and the plurality of first substances contained in the first liquid. The first separator 551 may deliver the first metal nanoparticle mass to the third chamber 33 and deliver the plurality of first substances to the fourth chamber 34. The first concentrator 561 may perform a concentration process on the first metal nanoparticle mass accommodated in the third chamber 33.

The second separator 552 may separate the second metal nanoparticle mass and the plurality of second substances contained in the second liquid. The second separator 552 may deliver the second metal nanoparticle mass to a sixth chamber 36 and deliver the plurality of second substances to a seventh chamber 37. The second concentrator 562 may perform a concentration process on the second metal nanoparticle mass accommodated in the sixth chamber 36. Through the concentration process of the second concentrator 562, at least one substance may be separated from the second metal nanoparticles and accommodated in an eighth chamber 38.

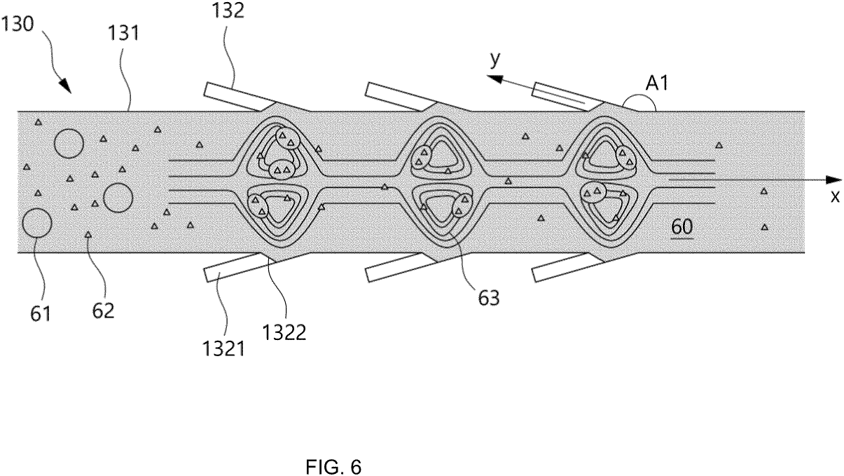

FIG. 6 is a view illustrating the filter according to an embodiment of the present disclosure.

Referring to FIG. 6, the filter 130 may include a first filter channel 131 and a fluid structure 132 formed to protrude outward from the first filter channel 131. The fluid structure 132 may form an obtuse angle with a direction x of the first filter channel 131. That is, an angle A1 between the direction x of the first filter channel 131 and a protruding direction y of the fluid structure 132 may be in a range of 90° to 180°.

A first liquid 60 flowing through the first filter channel 131 may include a first substance 61 and a second substance 62. The first substance 61 may be one of the blood cell components, and the second substance 62 may be one of the plasma components.

The fluid structure 132 may include an air bag 1321 that the first liquid 60 does not enter. The air bag 1321 may serve as a pump. The air bag 1321 may repeat contraction and expansion based on a first sound wave generated by the first vibration generator 133. Accordingly, the air bag 1321 may pump the first liquid 60 in the direction x of the first filter channel 131.

The fluid structure 132 may serve as a filter. When the first sound wave having a frequency corresponding to the size of the first substance 61 is generated from the first vibration generator 133, a vortex 63 of the first liquid 60 may be formed in a region near an interface 1322 between the first filter channel 131 and the air bag 1321. The first substance 61 may be trapped in the vortex 63. Accordingly, the fluid structure 132 may filter the first substance 61.

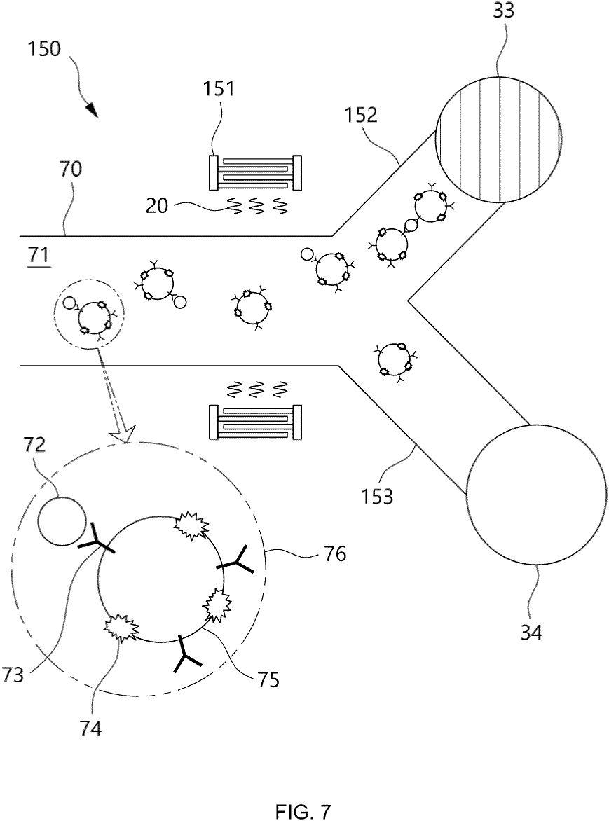

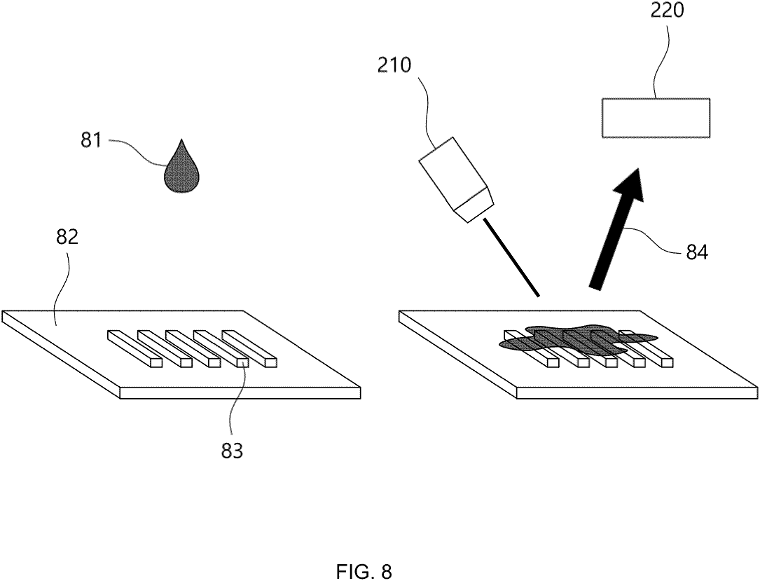

FIG. 7 is a view illustrating the separator according to an embodiment of the present disclosure.

Referring to FIG. 7, the separator 150 may include a channel 70 through which a liquid 71 flows and the second vibration generator 151 disposed at a side surface of the channel 70 to generate a sound wave. The separator 150 may include the first outlet channel 152 and the second outlet channel 153 branched from the channel 70. Although the separator 150 is illustrated as having a symmetrical structure in FIG. 7, the present disclosure is not limited thereto, and the separator 150 may also have an asymmetrical structure. The separator 150 may include three or more outlet channels.

The liquid 71 may contain a target substance 72, a reactant 73 which reacts with the target substance 72, and metal nanoparticles 75 to which the reactant 73 and Raman active molecules 74 are bound. A metal nanoparticle mass 76 may be formed due to an antigen-antibody reaction between the target substance 72 and the reactant 73. The target substance 72 may react with a plurality of reactants 73. The metal nanoparticle mass 76 may contain a plurality of metal nanoparticles.

The separator 150 may separate the plurality of substances contained in the liquid 71 according to the molecular weight based on the sound wave. The plurality of substances contained in the liquid 71 may be aligned according to the molecular weight upon encountering the sound wave. Here, the plurality of substances may move to form an acute angle with a direction of the channel 70.

The separator 150 may deliver a first substance whose molecular weight is higher than a predetermined value to the third chamber 33 through the first outlet channel 152. The separator 150 may deliver a second substance whose molecular weight is lower than the predetermined value to the fourth chamber 34 through the second outlet channel 153. The first substance may include the metal nanoparticle mass 76 formed due to the antigen-antibody reaction between the target substance 72 and the reactant 73. The second substance may include various substances excluding the target substance 72 or a substance to which the target substance 72 is bound. For example, the second substance may include the reactant 73 which does not react with the target substance 72. Alternatively, the second substance may include metal nanoparticles and Raman active molecules bound to the reactant 73 not reacting with the target substance 72.

FIG. 8 is a view for describing a liquid information obtaining method of a liquid information obtaining apparatus according to an embodiment of the present disclosure.

Referring to FIG. 8, a substrate 82 on which a liquid 81 is applied may be provided. The liquid 81 may be one of the liquids stored in the plurality of chambers 31, 32, and 33 of the liquid refining apparatus 100. A light source 210 of the liquid information obtaining apparatus 200 may irradiate the liquid 81 applied on the substrate 82 with light. A sensor 220 may sense light 84 scattered from fine particles contained in the liquid 81. The liquid information obtaining apparatus 200 may obtain spectral information on the liquid 81 based on the scattered light 84.

A metal pattern 83 may be formed on the substrate 82 for surface enhanced Raman scattering. The scattered light 84 sensed by the sensor 220 may be amplified due to the metal pattern 83.

A liquid information obtaining method may be different for the plurality of liquids collected from the plurality of chambers 31, 32, and 33. The first liquid stored in the first chamber 31 and the second liquid stored in the second chamber 32 may not contain metal nanoparticles for surface enhanced Raman scattering. Accordingly, when obtaining liquid information on the first liquid and the second liquid, the first liquid and the second liquid may be applied on the substrate 82 on which the metal pattern 83 is formed. The third liquid stored in the third chamber 33 may contain metal nanoparticles. The third liquid may be applied on the substrate 82 on which the metal pattern 83 is not formed. Alternatively, in order to further improve the effect of surface enhanced Raman scattering, the third liquid may be applied on the substrate 82 on which the metal pattern 83 is formed.

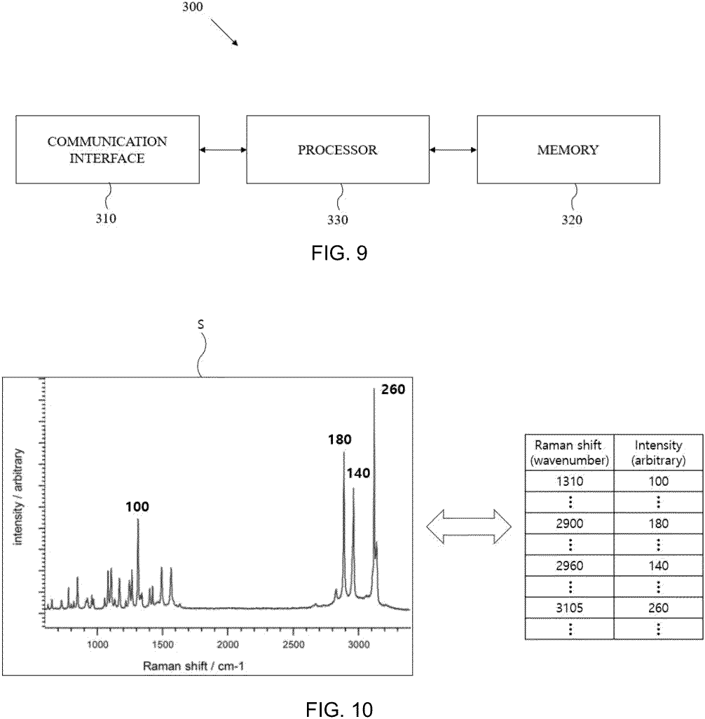

FIG. 9 is a block diagram illustrating a configuration of the diagnosis apparatus according to an embodiment of the present disclosure. Referring to FIG. 9, the diagnosis apparatus 300 may include a communication interface 310, a memory 320, and a processor 330.

The communication interface 310 may include at least one communication circuit and may perform communication with various types of external devices or external servers. For example, the communication interface 310 may receive information on a spectrum that corresponds to blood of a subject from an external apparatus.

The communication interface 310 may include at least one of a Wi-Fi communication module, a cellular communication module, a 3^{rd} generation (3G) mobile communication module, a 4^{th} generation (4G) mobile communication module, a 4G long term evolution (LTE) communication module, a 5^{th} generation (5G) mobile communication module, and a wired Ethernet.

The memory 320 may store an operating system (OS) for controlling the overall operation of the elements of the diagnosis apparatus 300 and commands or data related to the elements of the diagnosis apparatus 300. The memory 320 may store information on a plurality of neural network models. The information on the plurality of neural network models may include information on a parameter that corresponds to each of the plurality of neural network models and learning data for learning of the plurality of neural network models. The learning data may include labeled data (or ground truth) indicating a concentration corresponding to the intensity of a spectrum. The learning data may include labeled data indicating a diagnosis result corresponding to the intensity of a spectrum or the concentration. The memory 320 may be implemented using a nonvolatile memory (e.g., a hard disk, a solid state drive (SSD), a flash memory) or a volatile memory.

The processor 330 may be electrically connected to the memory 320 to control the overall function and operation of the diagnosis apparatus 300. The processor 330 may receive spectral information (or information on a spectrum) of a liquid collected from a subject, from an external apparatus through the communication interface 310. Alternatively, the processor 330 may obtain the spectral information through an input interface. The information on the spectrum may include a numerical value indicating the spectrum, a vector, and at least one peak value included in the spectrum. A frequency at which a peak value of the spectrum appears may correspond to Raman active molecules bound to metal nanoparticles bound to a target substance.

The processor 330 may obtain information on a spectrum of each of the plurality of liquids. For example, the processor 330 may obtain information on a spectrum that corresponds to a first liquid collected from a subject. The first liquid may be whole blood of the subject. The processor 330 may obtain information on a spectrum that corresponds to a second liquid obtained by reducing a concentration of blood cell components in the first liquid. The processor 330 may obtain information on a spectrum that corresponds to a third liquid obtained by adding a first reactant (e.g., a first antibody) for detection of a first target substance (e.g., a first antigen) into the second liquid. The processor 330 may obtain information on a spectrum that corresponds to a fourth liquid obtained by adding a second reactant (e.g., a second antibody) for detection of a second target substance (e.g., a second antigen) into the second liquid.

The processor 330 may obtain concentration information of a target substance based on the information on the spectrum. The concentration information of the target substance may include a numerical value indicating the concentration of the target substance, a feature vector corresponding to the concentration of the target substance, and a peak value of the spectrum corresponding to the concentration of the target substance.

The processor 330 may obtain first concentration information of a first target substance based on information on a first spectrum and information on a second spectrum. Here, the information on the first spectrum may be information on a first liquid which contains the first target substance and does not contain a first reactant reacting with the first target substance. The information on the second spectrum may be information on a second liquid obtained by adding the first reactant to the first liquid. For example, the first liquid may be a liquid accommodated in the second chamber 32 of FIG. 3. The second liquid may be a liquid accommodated in the third chamber 33 of FIG. 3. In the second liquid, the content of the remaining components excluding the first target substance (e.g., proteins excluding the first target substance) may be lower as compared to the first liquid. The remaining components may act as noise when obtaining information on a spectrum. Therefore, by using information on a spectrum for each of the first liquid from which the remaining components are not removed and the second liquid from which at least a portion of the remaining components are removed, the processor 330 may obtain the first concentration information in which noise due to the remaining components is reduced.

The processor 330 may obtain concentration information based on a lookup table in which the information on the second spectrum, a peak value of the spectrum, and the concentration information are matched. The lookup table may be pre-stored in the memory 320. For example, the processor 330 may identify concentration information that corresponds to a peak value of the second spectrum in the lookup table.

The processor 330 may compensate for the concentration information obtained based on the lookup table. For example, the processor 330 may input the information on the first spectrum into a first neural network model to obtain a coefficient for compensating for the concentration information. The processor 330 may compensate for the concentration information based on the coefficient to obtain the first concentration information of the first target substance.

The processor 330 may input the information on the first spectrum and the information on the second spectrum into a second neural network model to obtain the first concentration information on the first target substance. The second neural network model may be a model learned to obtain concentration information based on information on a spectrum.

The processor 330 may input the information on the second spectrum into the second neural network model to obtain a feature vector. The processor 330 may input the feature vector and the information on the first spectrum into a third neural network model to obtain the first concentration information. The third neural network model may be a model learned to obtain compensated concentration information based on information on a spectrum and concentration information. The feature vector may be obtained from an output end of a layer (e.g., a fully connected layer) included in the second neural network model.

The processor 330 may input the information on the first spectrum into the second neural network model to obtain a first feature vector. The processor 330 may input the information on the second spectrum into the second neural network model to obtain a second feature vector. The processor 330 may input the first feature vector and the second feature vector into a fourth neural network model to obtain the first concentration information. The fourth neural network model may be a model learned to obtain compensated concentration information based on the concentration information.

The processor 330 may obtain diagnosis information based on information on a spectrum and concentration information. For example, the processor 330 may input the information on the first spectrum and the first concentration information into a fifth neural network model to obtain diagnosis information. Alternatively, the processor 330 may input the information on the first spectrum, the first concentration information of the first target substance, and second concentration information of a second target substance into the fifth neural network model to obtain diagnosis information. The fifth neural network model may be a model learned to obtain diagnosis information based on spectral information and concentration information.

The processor 330 may obtain diagnosis information based on information on a spectrum that corresponds to each of a plurality of liquids. For example, the processor 330 may input the information on the first spectrum and the information on the second spectrum into a sixth neural network model to obtain diagnosis information. The sixth neural network model may be a model learned to obtain diagnosis information based on spectral information.

Meanwhile, an artificial intelligence-related function according to the present disclosure is operated through the processor 330 and the memory 320. The processor 330 may be formed of one or more processors. Here, the one or more processors may be a universal processor such as a central processing unit (CPU), an application processor (AP), and a digital signal processor (DSP), a dedicated graphics processor such as a graphics processing unit (GPU) and a vision processing unit (VPU), or a dedicated artificial intelligence processor such as a neural processing unit (NPU). The one or more processors control input data to be processed according to a predefined action rule or artificial intelligence model stored in the memory 320. Alternatively, in a case where the one or more processors are dedicated artificial intelligence processors, the dedicated artificial intelligence processors may be designed to have a hardware structure specialized for processing of a specific artificial intelligence model.

The predefined action rule or artificial intelligence model is formed through learning. Here, being formed through learning indicates that the predefined action rule or artificial intelligence model set to perform a desired characteristic (or purpose) is formed by a basic artificial intelligence model learning using a plurality of pieces of learning data by a learning algorithm. The learning may be performed by the device itself in which artificial intelligence according to the present disclosure is performed or may be performed through a separate server and/or system. Examples of the learning algorithm include supervised learning, unsupervised learning, semi-supervised learning, and reinforcement learning, but the learning algorithm is not limited to the above-listed examples.

An artificial intelligence model may be formed through learning. The artificial intelligence model may be formed of a plurality of neural network layers. The plurality of neural network layers each have a plurality of weight values and perform neural network operation through an operation between an operation result of the previous layer and the plurality of weight values. The plurality of weight values that the plurality of neural network layers have may be optimized by a learning result of the artificial intelligence model. For example, the plurality of weight values may be updated so that a loss value or cost value obtained from the artificial intelligence model during the learning process is reduced or minimized.

An artificial neural network may include a deep neural network (DNN). For example, the artificial neural network may include a convolutional neural network (CNN), a DNN, a recurrent neural network (RNN), a generative adversarial network (GAN), a restricted Boltzmann machine (RBM), a deep belief network (DBN), a bidirectional recurrent deep neural network (BRDNN), or a deep Q-network but is not limited to the above-listed examples.

FIG. 10 is a view illustrating information on a spectrum according to an embodiment of the present disclosure.

Referring to FIG. 10, information on a spectrum S (or spectral information) may indicate an intensity of a Raman signal according to a wavenumber. The spectral information S may be vector data in which wavenumbers and intensities of a Raman signal are matched with each other. In various embodiments described below, the spectral information S may be input into a neural network model. Here, the spectral information S input into a neural network model may include a plurality of intensities that respectively correspond to a plurality of wavenumbers. Alternatively, the spectral information S may indicate a single intensity that corresponds to a specific wavenumber (e.g., a wavenumber that corresponds to Raman active molecules).

The spectral information S may be normalized data. For example, a specific liquid may be irradiated with a laser beam having a first intensity to obtain a first Raman signal and may be irradiated with a laser beam having a second intensity to obtain a second Raman signal. Here, the diagnosis apparatus 300 may control the intensity of the first Raman signal or second Raman signal so that an area of the first Raman signal and an area of the second Raman signal become equal.

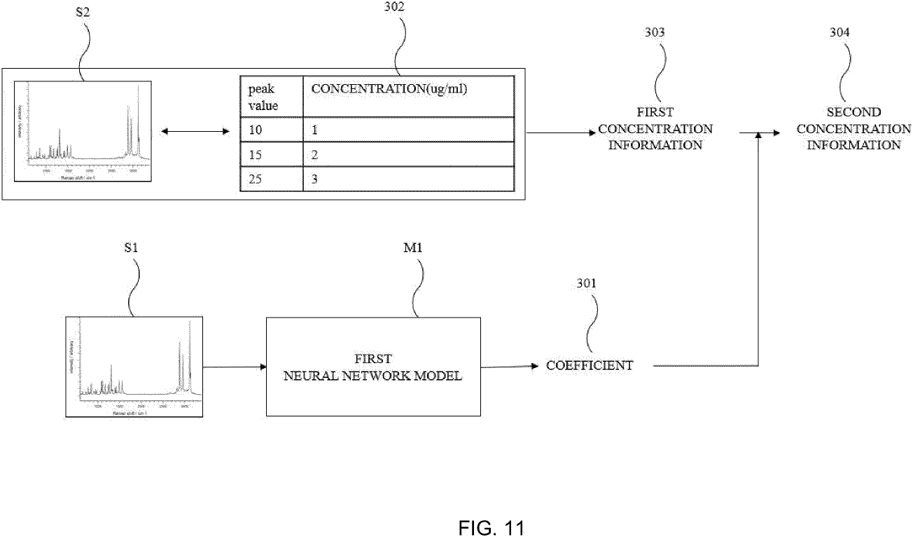

FIG. 11 is a view for describing a method of obtaining concentration information according to a first embodiment of the present disclosure.

Referring to FIG. 11, the diagnosis apparatus 300 may obtain information on a first spectrum S1 that corresponds to a first liquid and information on a second spectrum S2 that corresponds to a second liquid. The first liquid may contain a first target substance (e.g., an antigen A) and not contain a first reactant (e.g., an antibody A′) corresponding to the first target substance. The second liquid is a liquid obtained by adding the first reactant to the first liquid and may contain the first target substance bound to the first reactant.

The diagnosis apparatus 300 may obtain first concentration information 303 of the first target substance based on the information on the second spectrum S2 and a lookup table 302. The lookup table 302 may include peak vales of spectra and concentrations matched with each other. The diagnosis apparatus 300 may obtain a peak value of the second spectrum based on the information on the second spectrum S2. For example, the diagnosis apparatus 300 may identify an intensity corresponding to a predetermined wavenumber in the information on the second spectrum S2 as the peak value. Here, the predetermined wavenumber may correspond to Raman active molecules bound to metal nanoparticles bound to the first reactant.

The diagnosis apparatus 300 may obtain the first concentration information 303 based on the peak value, which is obtained from the information on the second spectrum S2, and the lookup table 302. For example, the diagnosis apparatus 300 may, from the lookup table 302, identify a concentration corresponding to the peak value obtained from the information on the second spectrum S2 and obtain the concentration as the first concentration information 303.

Meanwhile, the second liquid may contain various components (e.g., proteins or the like) other than the first target substance. Accordingly, the first concentration information 303 obtained based on the lookup table 302 may include an error.

The diagnosis apparatus 300 may compensate for the first concentration information 303 based on the information on the first spectrum S1. The diagnosis apparatus 300 may input the information on the first spectrum S1 into a first neural network model M1 to obtain a coefficient 301 for compensating for the concentration information. Here, the information on the first spectrum S1 may include a plurality of intensities according to a plurality of wavenumbers. The diagnosis apparatus 300 may compensate for the first concentration information 303 based on the coefficient 301. For example, the diagnosis apparatus 300 may multiply the first concentration information 303 by the coefficient 301 to obtain second concentration information 304.

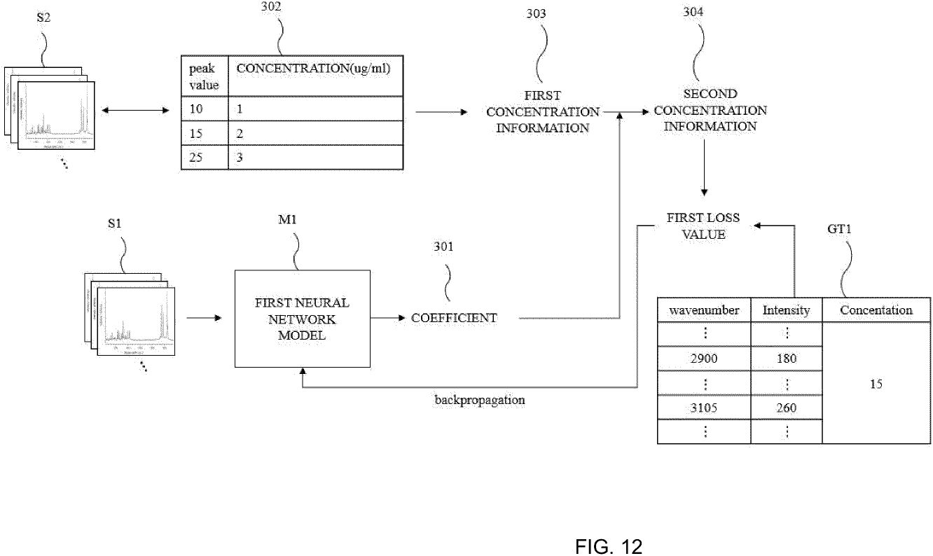

FIG. 12 is a view for describing a learning method of the first neural network model according to an embodiment of the present disclosure.

Referring to FIG. 12, learning data of the first neural network model M1 may include a plurality of pieces of first spectral information S1, a plurality of pieces of second spectral information S2, and a first ground truth GT1. The first ground truth GT1 may include target substance density information that corresponds to spectral information corresponding to a liquid containing a target substance. The first ground truth GT1 may be pre-stored in the memory 320 of the diagnosis apparatus 300.

The diagnosis apparatus 300 may cause the first neural network model M1 to learn based on the first ground truth GT1. The diagnosis apparatus 300 may obtain the second concentration information 304 based on the first spectral information S1 and the second spectral information S2. A method of obtaining the second concentration information 304 has been described above with reference to FIG. 11, and thus detailed description thereof will be omitted. The diagnosis apparatus 300 may obtain a first loss value based on the second concentration information 304 and concentration information included in the first ground truth GT1. The diagnosis apparatus 300 may update a parameter (e.g., a weight value) of the first neural network model M1 until the first loss value becomes smaller than a predetermined value. That is, the diagnosis apparatus 300 may cause the first neural network model M1 to learn based on backpropagation.

Meanwhile, although FIG. 12 illustrates an example in which the diagnosis apparatus 300 causes the first neural network model M1 to learn based on supervised learning, this is only an embodiment, and the diagnosis apparatus 300 may also cause the first neural network model M1 to learn based on unsupervised learning (e.g., reinforcement learning).

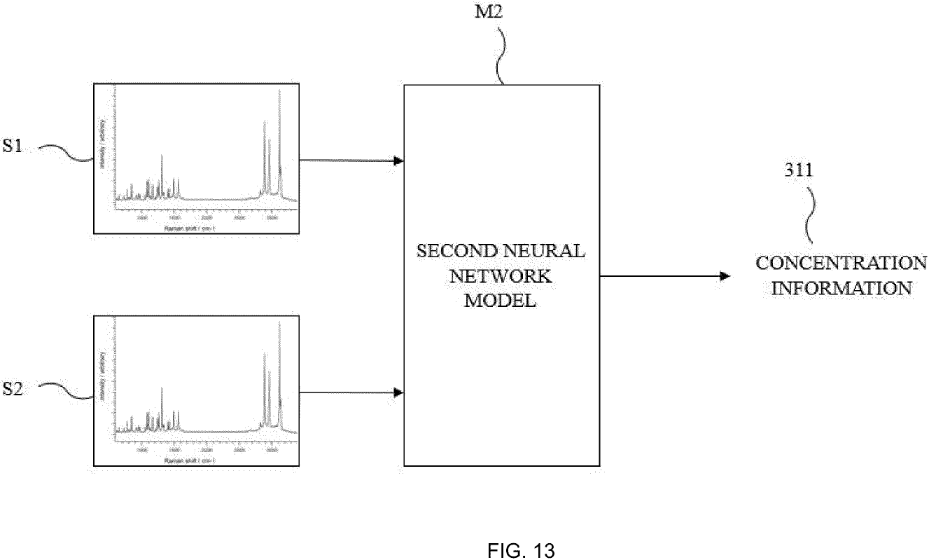

FIG. 13 is a view for describing a method of obtaining concentration information according to a second embodiment of the present disclosure.

Referring to FIG. 13, the diagnosis apparatus 300 may input the information on the first spectrum S1 and the information on the second spectrum S2 into a second neural network model M2 to obtain concentration information 311 of a first target substance. For example, the information on the second spectrum S2 may indicate a single intensity that corresponds to a specific wavenumber (e.g., a wavenumber that corresponds to Raman active molecules).

The first liquid and the second liquid may contain other components (e.g., blood cell components), excluding the first target substance, in common. The second neural network model M2 may obtain the concentration information 311 based on spectral information that corresponds to a plurality of liquids containing the other components in common, instead of obtaining the concentration information 311 only based on spectral information that corresponds to a single liquid. Therefore, the accuracy of the concentration information 311 may be improved.

The diagnosis apparatus 300 may cause the second neural network model M2 to learn. Learning data of the second neural network model M2 may include the plurality of pieces of first spectral information S1, the plurality of pieces of second spectral information S2, and a second ground truth. The second ground truth may include concentration information that corresponds to spectral information. The diagnosis apparatus 300 may obtain a second loss value based on the concentration information 311 and the concentration information included in the second ground truth. The diagnosis apparatus 300 may update a parameter (e.g., a weight value) of the second neural network model M2 until the second loss value becomes smaller than a predetermined value.

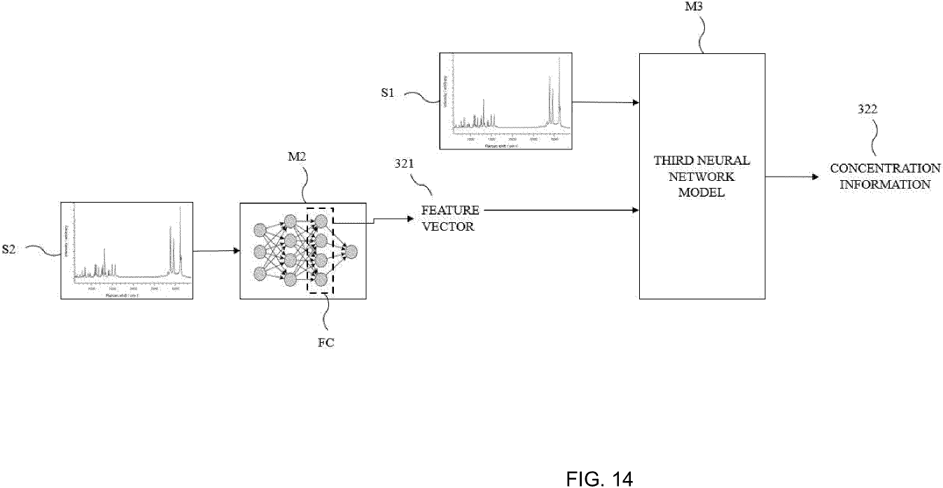

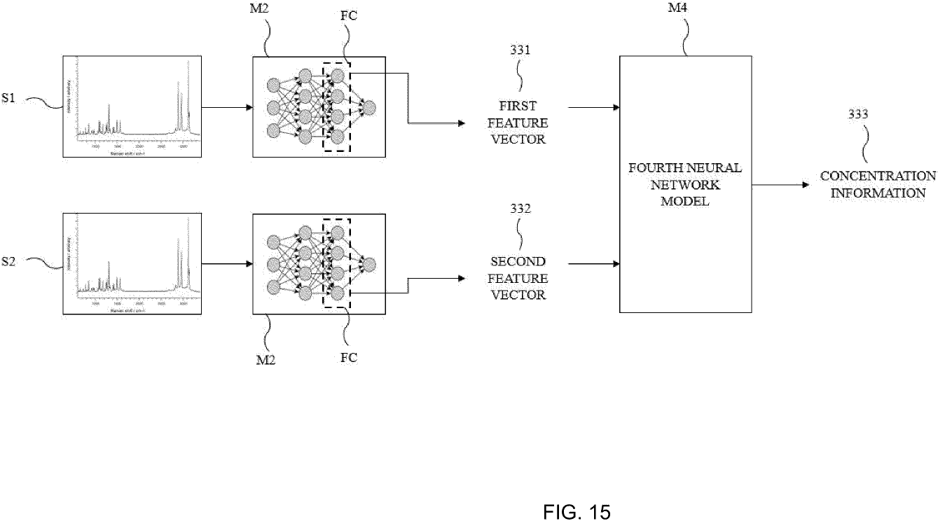

FIG. 14 is a view for describing a method of obtaining concentration information according to a third embodiment of the present disclosure.

Referring to FIG. 14, the diagnosis apparatus 300 may input the information on the second spectrum S2 into the second neural network model M2 to obtain a feature vector 321. The feature vector 321 may be output through a fully connected layer FC of the second neural network model M2. The diagnosis apparatus 300 may input the information on the first spectrum S1 and the feature vector 321 into a third neural network model M3 to obtain concentration information 322.

The diagnosis apparatus 300 may cause the third neural network model M3 to learn. Learning data of the third neural network model M3 may include the plurality of pieces of first spectral information S1, the plurality of pieces of second spectral information S2, and a third ground truth. The third ground truth may include concentration information that corresponds to spectral information and a feature vector. The diagnosis apparatus 300 may obtain a third loss value based on the concentration information 322 and the concentration information included in the third ground truth. The diagnosis apparatus 300 may update a parameter (e.g., a weight value) of the third neural network model M3 until the third loss value becomes smaller than a predetermined value.

The diagnosis apparatus 300 may also cause the second neural network model M2 to learn while causing the third neural network model M3 to learn. The diagnosis apparatus 300 may update a parameter of the second neural network model M2 until the third loss value becomes smaller than the predetermined value. Alternatively, the diagnosis apparatus 300 may cause the third neural network model M3 to learn after learning of the second neural network model M2 is completed. Here, the parameter of the second neural network model M2 may be frozen while the parameter of the third neural network model M3 is being updated.

FIG. 15 is a view for describing a method of obtaining concentration information according to a fourth embodiment of the present disclosure.

Referring to FIG. 15, the diagnosis apparatus 300 may input the information on the first spectrum S1 into the second neural network model M2 to obtain a first feature vector 331. The diagnosis apparatus 300 may input the information on the second spectrum S2 into the second neural network model M2 to obtain a second feature vector 332. The diagnosis apparatus 300 may input the first feature vector 331 and the second feature vector 332 into a fourth neural network model M4 to obtain concentration information 333.

The diagnosis apparatus 300 may cause the fourth neural network model M4 to learn. Learning data of the fourth neural network model M4 may include the plurality of pieces of first spectral information S1, the plurality of pieces of second spectral information S2, and a fourth ground truth. The fourth ground truth may include concentration information that corresponds to a feature vector. The diagnosis apparatus 300 may obtain a fourth loss value based on the concentration information 333 and the concentration information included in the fourth ground truth. The diagnosis apparatus 300 may update a parameter (e.g., a weight value) of the fourth neural network model M4 until the fourth loss value becomes smaller than a predetermined value.

The diagnosis apparatus 300 may also cause the second neural network model M2 to learn while causing the fourth neural network model M4 to learn. The diagnosis apparatus 300 may update the parameter of the second neural network model M2 until the fourth loss value becomes smaller than the predetermined value. Alternatively, the diagnosis apparatus 300 may cause the fourth neural network model M4 to learn after learning of the second neural network model M2 is completed. Here, the parameter of the second neural network model M2 may be frozen while the parameter of the fourth neural network model M4 is being updated.

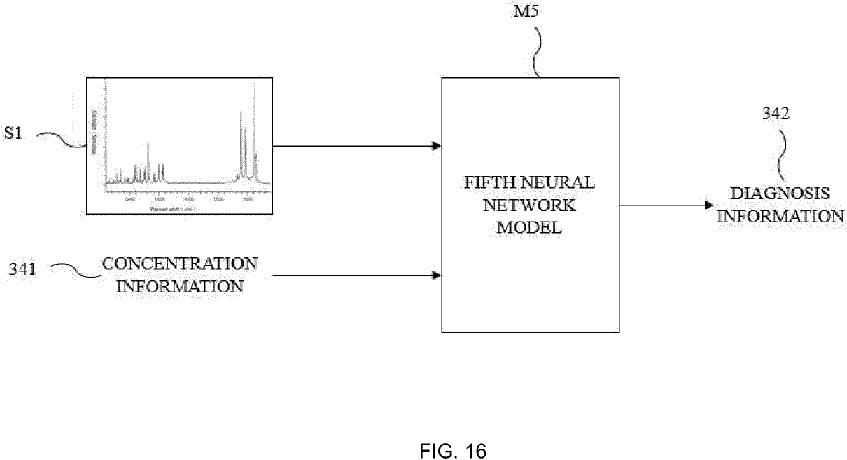

FIG. 16 is a view for describing a method of obtaining concentration information according to an embodiment of the present disclosure.

Referring to FIG. 16, the diagnosis apparatus 300 may input the information on the first spectrum S1 and concentration information 341 of a first target substance into a fifth neural network model M5 to obtain diagnosis information 342. For example, the diagnosis information 342 may include information on a disease that the subject is expected to have. Here, the first target substance may be a biomarker of the disease.

The diagnosis apparatus 300 may cause the fifth neural network model M5 to learn. Learning data of the fifth neural network model M5 may include the plurality of pieces of first spectral information S1, a plurality of pieces of concentration information 341, and a fifth ground truth. The plurality of pieces of concentration information 341 may be obtained according to various embodiments described above. The fifth ground truth may include diagnosis information that corresponds to spectral information and concentration information.

The diagnosis apparatus 300 may obtain a fifth loss value based on the diagnosis information 342 and the diagnosis information included in the fifth ground truth. The diagnosis apparatus 300 may update a parameter (e.g., a weight value) of the fifth neural network model M5 until the fifth loss value becomes smaller than a predetermined value. The diagnosis apparatus 300 may cause a neural network model outputting the concentration information 341 to learn while causing the fifth neural network model M5 to learn. For example, in a case where the concentration information 341 is obtained by the second neural network model M2, the diagnosis apparatus 300 may update the parameter of the second neural network model M2 until the fifth loss value becomes smaller than a predetermined value. Alternatively, the diagnosis apparatus 300 may cause the fifth neural network model M5 to learn after learning of the neural network model outputting the concentration information 341 is completed.

Meanwhile, disease diagnosis may be performed based on a single biomarker but may also be performed using a plurality of biomarkers.

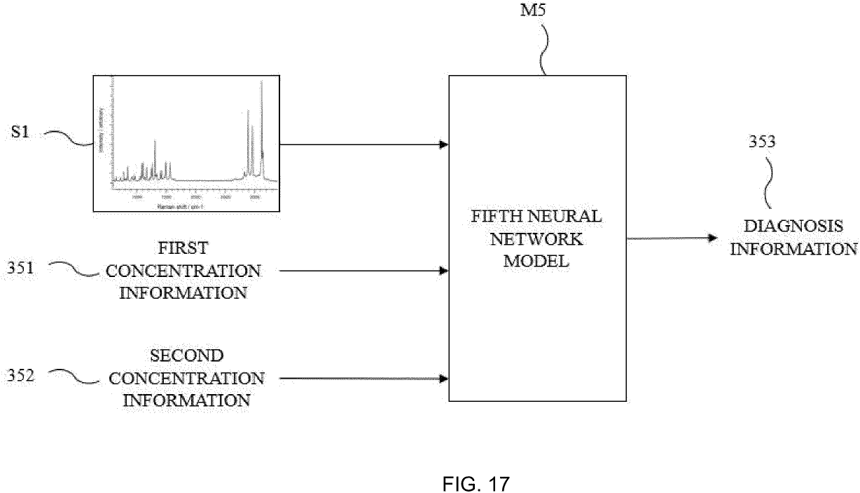

FIG. 17 is a view for describing a method of obtaining concentration information according to an embodiment of the present disclosure.

Referring to FIG. 17, the diagnosis apparatus 300 may input the information on the first spectrum S1, first concentration information 351 of a first target substance, and second concentration information 352 of a second target substance into the fifth neural network model M5 to obtain diagnosis information 353. For example, the diagnosis information 353 may include a diagnosis result relating to Alzheimer’s disease. The first target substance may be tau protein, and the second target substance may be β-amyloid.

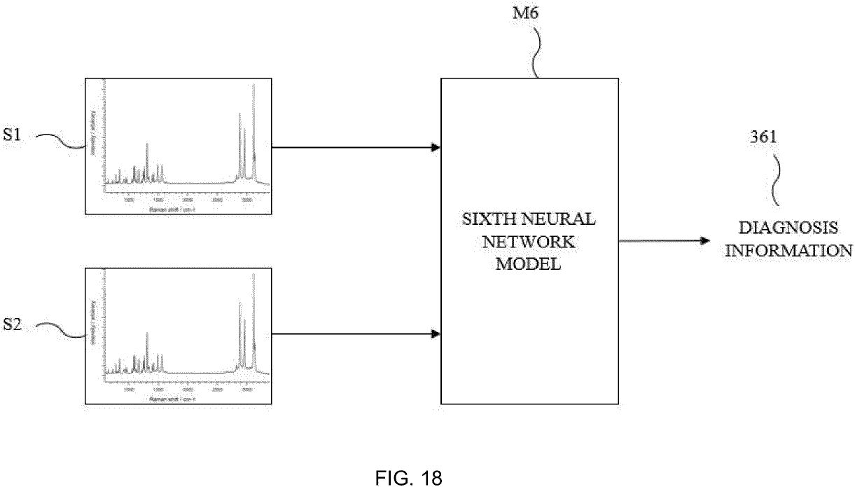

FIG. 18 is a view for describing a method of obtaining concentration information according to an embodiment of the present disclosure.

Referring to FIG. 18, the diagnosis apparatus 300 may input the information on the first spectrum S1 and the information on the second spectrum S2 into a sixth neural network model M6 to obtain diagnosis information 361. The information on the first spectrum S1 may correspond to a first liquid containing a first target substance. The information on the second spectrum S2 may correspond to a second liquid containing the first target substance and a first reactant bound to the first target substance.

The diagnosis apparatus 300 may cause the sixth neural network model M6 to learn. Learning data of the sixth neural network model M6 may include the plurality of pieces of first spectral information S1, the plurality of pieces of second spectral information S2, and a sixth ground truth. The sixth ground truth may include diagnosis information that corresponds to spectral information. The diagnosis apparatus 300 may obtain a sixth loss value based on the diagnosis information 361 and the diagnosis information included in the sixth ground truth. The diagnosis apparatus 300 may update a parameter (e.g., a weight value) of the sixth neural network model M6 until the sixth loss value becomes smaller than a predetermined value.

Preprocessing may be performed for input data input into the plurality of neural network models M1, M2, M3, M4, M5, and M6 according to the present disclosure. For example, concatenation may be performed for a plurality of pieces of input data.

Some of the plurality of neural network models M1, M2, M3, M4, M5, and M6 may be integrated.

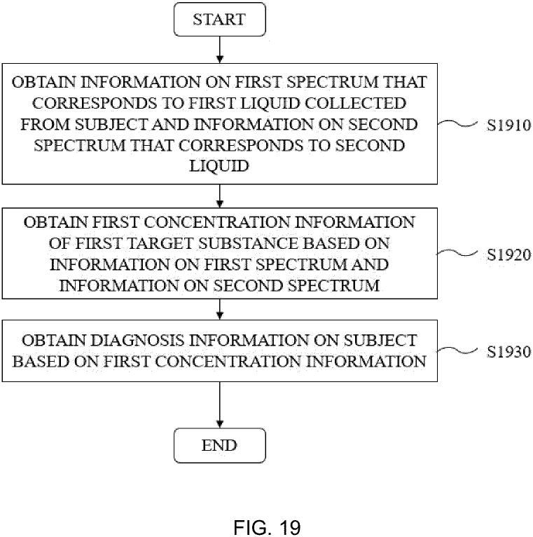

FIG. 19 is a flowchart illustrating a control method of the diagnosis apparatus according to an embodiment of the present disclosure.

Referring to FIG. 19, the diagnosis apparatus 300 may obtain information on a first spectrum that corresponds to a first liquid collected from a subject and information on a second spectrum that corresponds to a second liquid (S1910). The first liquid may contain a first target substance. The second liquid may be a liquid obtained by adding a first reactant corresponding to the first target substance to the first liquid. The second liquid may contain the first target substance bound to the first reactant.

The diagnosis apparatus 300 may obtain first concentration information of the first target substance based on the information on the first spectrum and the information on the second spectrum (S1920). For example, the diagnosis apparatus 300 may obtain concentration information based on the information on the second spectrum and a lookup table in which peak values of spectra and pieces of concentration information are matched. The diagnosis apparatus 300 may input the information on the first spectrum into a first neural network model to obtain a coefficient for compensating for the concentration information. The diagnosis apparatus 300 may perform compensation for the concentration information based on the coefficient to obtain compensated concentration information.

The diagnosis apparatus 300 may input the information on the first spectrum and the information on the second spectrum into a second neural network model to obtain the first concentration information.

The diagnosis apparatus 300 may input the information on the second spectrum into the second neural network model to obtain a feature vector. The diagnosis apparatus 300 may input the information on the first spectrum and the feature vector into a third neural network model to obtain the first concentration information.

The diagnosis apparatus 300 may input the information on the first spectrum into the second neural network model to obtain a first feature vector. The diagnosis apparatus 300 may input the information on the second spectrum into the second neural network model to obtain a second feature vector. The diagnosis apparatus 300 may input the first feature vector and the second feature vector into a fourth neural network model to obtain the first concentration information.

The diagnosis apparatus 300 may obtain diagnosis information on the subject based on the first concentration information (S1930). The diagnosis apparatus 300 may input the information on the first spectrum and the first concentration information into a fifth neural network model to obtain diagnosis information.

The diagnosis apparatus 300 may obtain information on a third spectrum that corresponds to a third liquid obtained by adding a second reactant to the first liquid. The third liquid may contain a second target substance which is bound to the second reactant. The diagnosis apparatus 300 may obtain second concentration information of the second target substance based on the information on the first spectrum and the information on the third spectrum. The diagnosis apparatus 300 may input the information on the first spectrum, the first concentration information, and the second concentration information into the fifth neural network model to obtain diagnosis information.

The diagnosis apparatus 300 may input the information on the first spectrum and the information on the second spectrum into a sixth neural network model to obtain diagnosis information.

According to various embodiments of the present disclosure described above, an error in spectral information on a liquid collected from a subject can be reduced. A diagnosis apparatus can compensate for the error in the spectral information. Accordingly, the accuracy in a diagnosis result for the subject can be improved.

Other effects obtainable or predictable from the embodiments of the present disclosure have been disclosed directly or implicitly in the detailed description of the embodiments of the present disclosure. For example, various effects predictable according to the embodiments of the present disclosure have been disclosed in the detailed description given above.

Other aspects, advantages, and salient features of the present disclosure should be apparent to those of ordinary skill in the art from the detailed description above which discloses various embodiments of the present disclosure with reference to the accompanying drawings.

Various embodiments described above may be implemented in a recording medium that is readable by a computer or a device similar thereto, by using software, hardware or a combination thereof. In some cases, the embodiments described herein may be implemented as a processor itself. In a case where the embodiments are implemented as software, the embodiments such as procedures and functions described herein may be implemented as separate software modules. Each of the software modules may perform one or more functions and operations described herein.

Computer instructions for performing processing operations according to various embodiments of the present disclosure described above may be stored in a non-transitory computer-readable medium. Computer instructions stored in such a non-transitory computer-readable medium may cause a specific machine to perform the processing operations according to various embodiments described above, when the instructions are executed by the processor of the specific machine.

A non-transitory computer-readable medium refers to a medium that stores data semi-permanently and is readable by machines, instead of a medium that stores data for a short moment such as a register, a cache, and a memory. Specific examples of a non-transitory computer-readable medium may include a compact disc (CD), a digital versatile disc (DVD), a hard disc, a blue-ray disc, a universal serial bus (USB), a memory card, a read-only memory (ROM), and the like.

The machine-readable storage medium may be provided in the form of a non-transitory storage medium. Here, the term “non-transitory storage medium” only indicates that the storage medium is a tangible device and does not include signals (e.g., electromagnetic waves), and the term does not differentiate between a case in which data is semi-permanently stored in a storage medium and a case in which data is temporarily stored in a storage medium. For example, the “non-transitory storage medium” may include a buffer in which data is temporarily stored.

A method according to various embodiments that is disclosed in this document may be provided by being included in a computer program product. The computer program product may be traded as a commodity between a seller and a buyer. The computer program product may be distributed in the form of a machine-readable storage medium (e.g., a compact disc read-only memory (CD-ROM)) or may be distributed online directly (e.g., downloaded or uploaded) through an application store (e.g., the Play Store^{®}) or between two user devices (e.g., smartphones). In the case of online distribution, at least a portion of the computer program product (e.g., a downloadable application) may be at least temporarily stored in a machine-readable storage medium such as a server of a manufacturer, a server of an application store, or a memory of a relay server or may be temporarily generated.

Exemplary embodiments of the present disclosure have been illustrated and described above, but the present disclosure is not limited to the specific embodiments described above. Various modifications may be made to the above embodiments by those of ordinary skill in the art to which the present disclosure pertains without departing from the gist of the present disclosure claimed in the claims, and such modifications should not be individually understood from the technical spirit or outlook of the present disclosure.

What is claimed is:

1. A liquid refining apparatus comprising:
a substrate;
a loader which is formed on the substrate and configured to receive a first liquid;
a filter configured to reduce a concentration of at least one substance included in the first liquid to obtain a second liquid with a reduced concentration of the at least one substance;
a reactor configured to mix the second liquid with a reactant for target substance detection to obtain a third liquid including, among a plurality of substances included in the second liquid, a first substance which undergoes a predetermined reaction with the reactant and a second substance which does not undergo the predetermined reaction with the reactant; and
a separator configured to separate the first substance and the second substance.

2. The liquid refining apparatus of claim 1, wherein:
the first liquid contains blood in an undiluted state; and
the at least one substance includes blood cell components.

3. The liquid refining apparatus of claim 1, wherein the predetermined reaction includes an antigen-antibody reaction.

4. The liquid refining apparatus of claim 1, further comprising an anticoagulator configured to add an anticoagulant to the first liquid.

5. The liquid refining apparatus of claim 1, wherein the reactor includes:
a reactant storage configured to store the reactant; and
a zigzag mixing channel configured to increase a mixing rate of the second liquid and the reactant.

6. The liquid refining apparatus of claim 1, wherein the reactant is bound to metal nanoparticles which are bound to Raman active particles.

7. The liquid refining apparatus of claim 1, further comprising:
a first chamber formed on the substrate and connected to the loader to store the first liquid;
a second chamber formed on the substrate and connected to the filter to store the second liquid; and
a third chamber formed on the substrate and connected to the separator to store the first substance.

8. The liquid refining apparatus of claim 1, further comprising a concentrator configured to perform a concentration process on the third liquid,
wherein the concentration process includes at least one of drying, heating, and baking.

9. The liquid refining apparatus of claim 1, further comprising a pumper configured to move the first liquid, the second liquid, and the third liquid,
wherein the pumper includes at least one of a pneumatic pump, a vibration pump, a mechanical pump, and a capillary pump.

10. The liquid refining apparatus of claim 1, wherein the reactor includes:
a first reactor which includes a first reactant storage configured to store a first reactant for first target detection and a first mixing channel configured to increase a mixing rate of the second liquid and the first reactant;
a second reactor which includes a second reactant storage configured to store a second reactant for second target detection and a second mixing channel configured to increase a mixing rate of the second liquid and the second reactant;
a first channel configured to transfer the second liquid to the first reactor; and
a second channel configured to transfer the second liquid to the second reactor.

11. The liquid refining apparatus of claim 10, wherein the first channel and the second channel have a structure branched from an output end of the filter.

12. The liquid refining apparatus of claim 10, wherein the separator includes:
a first separator which is connected to an output end of the first reactor and configured to separate, among a plurality of substances contained in the third liquid, a third substance which undergoes an antigen-antibody reaction with the first reactant from another substance; and
a second separator which is connected to an output end of the second reactor and configured to separate, among the plurality of substances contained in the third liquid, a fourth substance which undergoes an antigen-antibody reaction with the second reactant from another substance.

13. The liquid refining apparatus of claim 1, wherein the filter includes a lateral cavity acoustic transducer (LCAT) formed to protrude outward from a filter channel through which the first liquid flows.

14. The liquid refining apparatus of claim 1, wherein the separator separates the first substance and the second substance according to the molecular weight based on a sound wave and includes a first outlet channel configured to move the first substance and a second outlet channel configured to move the second substance.

15. A diagnosis system including a liquid refining apparatus, a liquid information obtaining apparatus, and a diagnosis apparatus,
wherein the liquid refining apparatus includes a substrate, a loader which is formed on the substrate and configured to receive a first liquid collected from a subject, a filter which is configured to reduce a concentration of at least one substance contained in the first liquid to obtain a second liquid with a reduced concentration of the at least one substance, a reactor which is configured to mix the second liquid with a reactant for target substance detection to obtain a third liquid containing, among a plurality of substances contained in the second liquid, a first substance which undergoes a predetermined reaction with the reactant and a second substance which does not undergo the predetermined reaction with the reactant, and a separator which is configured to separate the first substance and the second substance,
the liquid information obtaining apparatus irradiates the first substance with light to obtain a Raman signal for the first substance, and
the diagnosis apparatus obtains a diagnosis result for the subject based on the Raman signal.
